(19) United States
(12) Patent Application Publication
(10) Pub. No.: US 2010/0099192 A1
(43) Pub. Date: Apr. 22, 2010

(54) TEST-SENSOR CARTRIDGE

(75) Inventors: Allen J. Brenneman, Goshen, IN (US); Huan-Ping Wu, Granger, IN (US)

Correspondence Address: NIXON PEABODY LLP, 300 S. Riverside Plaza, 16th Floor, CHICAGO, IL, 60606-6613, US

(73) Assignee: Bayer HealthCare, LLC, Tarrytown, NY (US)

(21) Appl. No.: 12/530,695

(22) PCT Filed: Mar. 12, 2007
(86) PCT No.: PCT/US07/06272

§ 371 Date: Sep. 10, 2009

(51) Int. Cl. G01N 33/487; B01L 9/00; G01N 35/00

(52) U.S. Cl. 436/43; 422/104; 435/287.1; 422/58

(57) ABSTRACT

A test-sensor cartridge is disclosed (200). The drum-like cartridge comprises a first face (202), a second opposing face (213), and a side portion (204) connecting the first and second opposing faces. With a plurality of test-sensor cavities (209) that is generally uniformly positioned therethrough. The plurality of the test sensor cavities are arranged in a non-radial layout. The plurality of test-sensor cavities contains a respective test sensor (207). The test sensor is adapted to assist in determining a concentration of an analyte, especially glucose.

### FIELD OF THE INVENTION

The present invention relates generally to analyte-testing instruments and, more particularly, to a test-sensor cartridge having a non-radial test-sensor layout.

### BACKGROUND OF THE INVENTION

The quantitative determination of analytes in body fluids is of great importance in the diagnoses and maintenance of certain physiological abnormalities. For example, lactate, cholesterol, and bilirubin should be monitored in certain individuals. In particular, determining glucose in body fluids is important to diabetic individuals who must frequently check the glucose level in their body fluids to regulate the glucose intake in their diets. The results of such tests may be used to determine what, if any, insulin and/or other medication needs to be administered. In one type of testing system, test sensors are used to test a fluid such as a sample of blood.

One method of monitoring an individual's blood glucose level is with a portable, hand-held blood glucose testing device (e.g., a meter). To determine the blood glucose level with the meter, a lancet device may be used with a needle lancet that pierces the skin tissue and allows a whole blood sample to form on the skin's surface. Once the requisite amount of blood forms on the skin's surface, the blood sample is transferred to a test sensor. The test sensor is generally placed in an opening in the body of the meter.

Test-sensor cartridges are commonly used to individually dispense test sensors to be used for testing an analyte in a fluid. Test-sensor cartridges may be incorporated directly into, for example, glucose meters to dispense test sensors for use with the meter. The cartridges are used to store multiple sensors and allow users to carry multiple sensors around within a single enclosure. The cartridges also assist in preventing or inhibiting the sensors from being exposed to the environment until they are required for use. A blood or body fluid sample may then be placed on the sensor and analyzed with the meter or similar device to determine the concentration of the analyte being examined.

One example of a prior art cartridge 100 is shown in FIGS. 1a,b. The prior art test-sensor cartridge 100 has a radial layout. In the illustrated embodiment, the cartridge 100 includes test-sensor cavities 108 arranged along radii of a flat face 102 of the cartridge 100. The cartridge 100 further includes ten test sensors 106, each of which is stored within a respective one of the corresponding ten test-sensor cavities 108.

Each time analyte-testing is performed, a new test sensor is used, and thus, a number of test sensors may be used in a single day. It is desirable for the meter to be generally compact in size so that the meter can be easily transported by a user. Existing meters contain one test-sensor cartridge. This may be undesirable since a user may realize that he or she has used all of the test sensors in the test-sensor cartridge at inopportune times. For example, the user may run out of test sensors while away from home and without having an extra cartridge available thereby likely causing inconvenience to the user.

Furthermore, existing test sensor cartridges include test sensors and test-sensor cavities arranged in a generally radial layout. To maximum the amount of test sensors included within the cartridge, the area between test sensors is often small. Thus, the area between the test sensors may include a thin section made of, for example, plastic or other suitable materials. The thin plastic section may be susceptible to tearing, or moisture may migrate from a cavity from which a sensor has been removed to the next sensor cavity via the thin plastic section. The thin plastic section may also make manufacturing of the cartridges difficult.

It would be desirable to have test-sensor cartridges that assist in addressing one or more of the above disadvantages.

### SUMMARY OF THE INVENTION

According to one embodiment of the present invention, a test-sensor cartridge comprises a first face, a second opposing face, and a side portion connecting the first and second opposing faces. The first face forms a plurality of test-sensor cavities that is generally uniformly positioned therethrough. The plurality of the test sensor cavities are arranged in a non-radial layout. The plurality of test-sensor cavities contains a respective test sensor. The test sensor is adapted to assist in determining a concentration of an analyte.

According to another embodiment of the present invention, an instrument adapted to determine an analyte concentration of a fluid sample using a test sensor is disclosed. The instrument comprises a display adapted to display information to a user and a user-interface mechanism adapted to allow the user to interact with the instrument. The instrument further comprises a test-sensor cartridge comprising a first face, a second opposing face, and a side portion connecting the first and second opposing faces. The first face forms a plurality of test-sensor cavities that is generally uniformly positioned therethrough. The plurality of the test sensor cavities is arranged in a non-radial layout. The plurality of test-sensor cavities contains a respective test sensor. The test sensor is adapted to assist in determining a concentration of the analyte. The instrument further comprises a body portion including at least a first opening formed therein. The first opening is adapted to receive a test sensor from the test-sensor cartridge.

According to another embodiment of the present invention, a method of excising a test sensor from a test-sensor cartridge located within an analyte-testing instrument is disclosed. The method comprises the act of providing a test-sensor cartridge comprising a first face, a second opposing face, and a side portion connecting the first and second opposing faces. The first face forms a plurality of test-sensor cavities that is generally uniformly positioned therethrough. The plurality of the test sensor cavities are arranged in a non-radial layout. The plurality of test-sensor cavities contain a respective test sensor. The test sensor is adapted to assist in determining a concentration of an analyte. The method further comprises the act of providing an instrument comprising a display adapted to display information to a user, a user-interface mechanism adapted to allow the user to interact with the instrument, a body portion including at least a first opening formed therein, the first opening being adapted to receive a test sensor from the test-sensor cartridge, and an excise mechanism. The method further comprises the act of moving the excise mechanism so as to excise a test sensor from the cartridge, the excised test sensor being positioned within the first opening of the instrument.

The above summary of the present invention is not intended to represent each embodiment or every aspect of the present invention. Additional features and benefits of the present invention are apparent from the detailed description and figures set forth below.

### BRIEF DESCRIPTION OF THE DRAWINGS

FIG. 1a is a front view of a prior art test-sensor cartridge according to one embodiment.

FIG. 1b is a side perspective view of the prior art test-sensor cartridge of FIG. 1a.

FIG. 2a is a front view of a test-sensor cartridge according to one embodiment of the present invention.

FIG. 2b is a side perspective view of the test-sensor cartridge of FIG. 2a.

FIG. 2c is a side perspective view of the test-sensor cartridge of FIGS. 2a,b.

FIG. 2d is an exploded side view of the test-sensor cartridge of FIG. 2c.

FIG. 3a is a front view of a test-sensor cartridge according to another embodiment of the present invention.

FIG. 3b is a side perspective view of the test-sensor cartridge of FIG. 3a.

FIG. 4 is a side perspective view of an analyte-testing instrument according to one embodiment of the present invention.

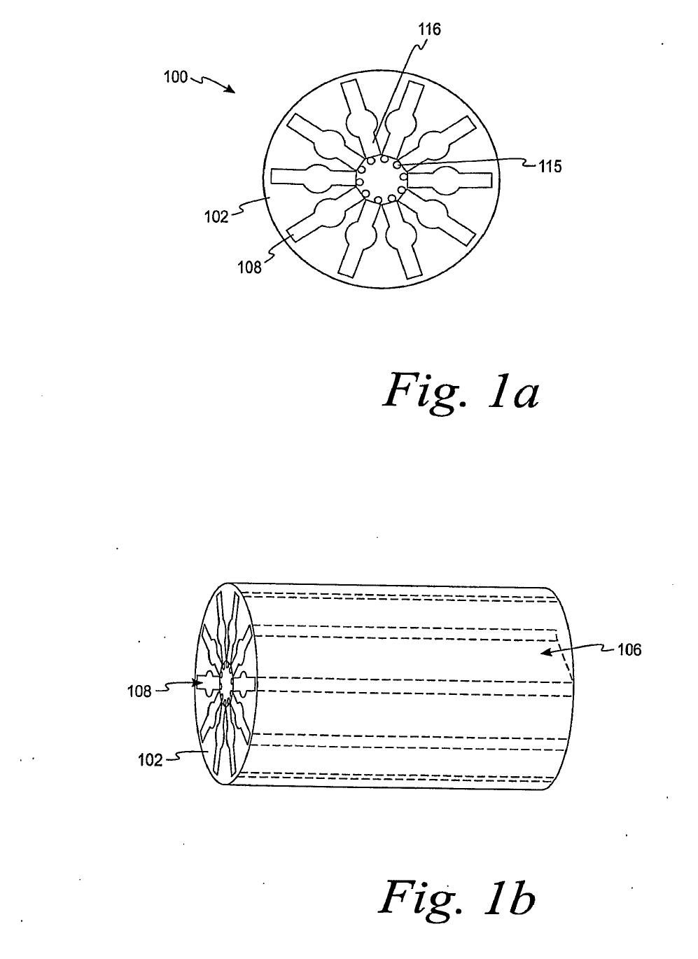

### DESCRIPTION OF ILLUSTRATED EMBODIMENTS

The present invention is directed to test-sensor cartridges. The test sensors (e.g., biosensors) excised from the cartridge may be used to assist in determining an analyte concentration in a fluid sample. Some examples of the types of analytes that may be collected and analyzed include glucose, lipid profiles (e.g., cholesterol, triglycerides, LDL, and HDL), microalbumin, fructose, lactate, or bilirubin. The present invention is not limited, however, to these specific analytes, and it is contemplated that other analyte concentrations may be determined. The analytes may be in, for example, a whole blood sample, a blood serum sample, a blood plasma sample, or other body fluids like ISF (interstitial fluid) and/or urine. One non-limiting example of the analyte-testing instruments' use is to determine the glucose concentration in a user's blood, plasma, or ISF.

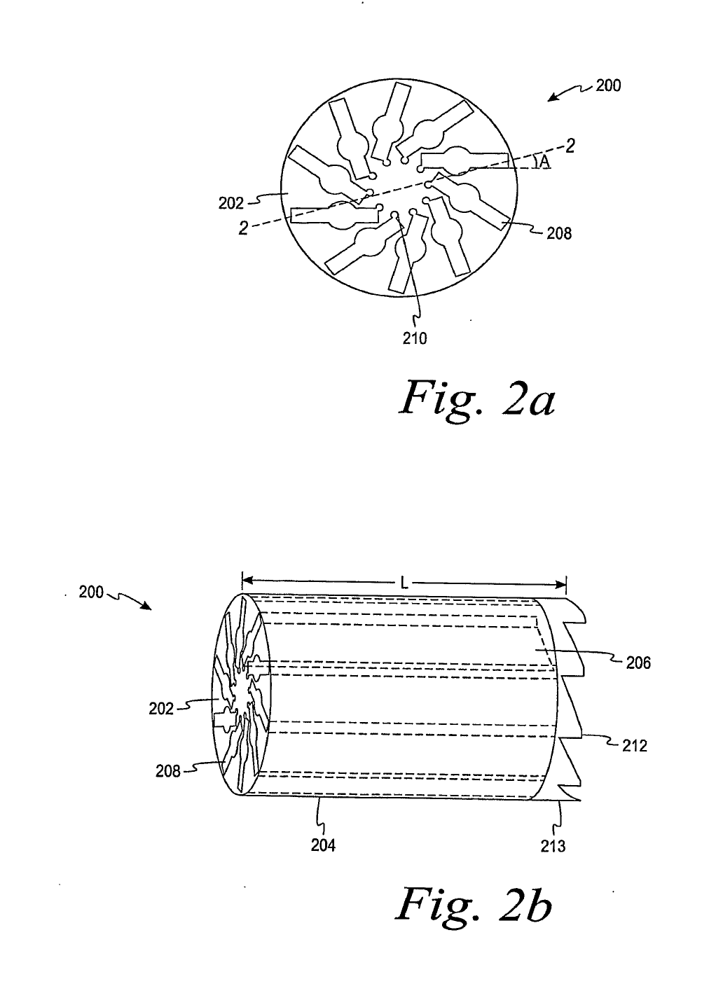

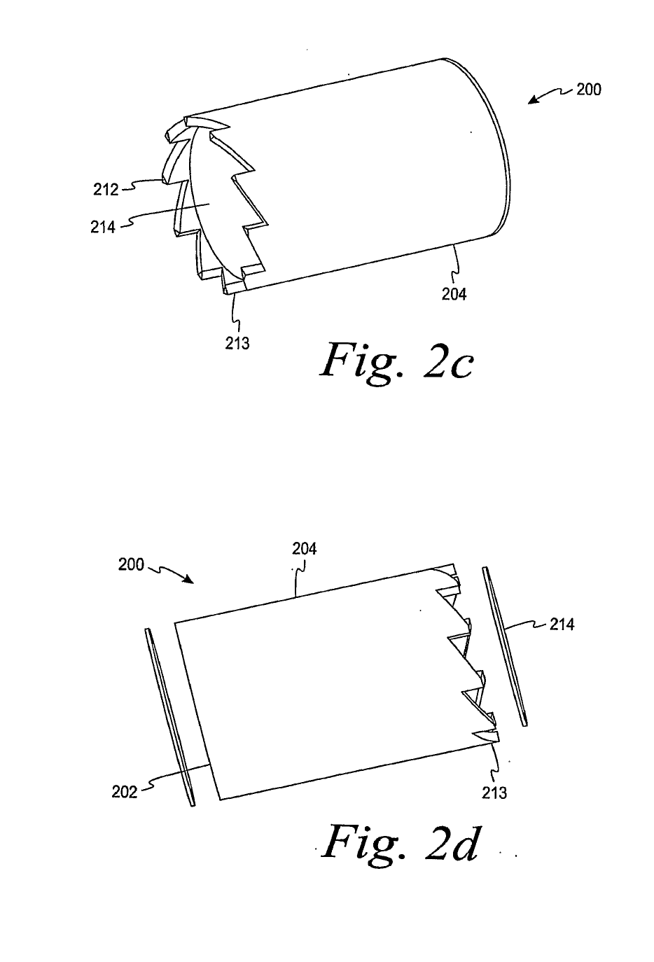

Turning now to the drawings and initially to FIGS. 2a,b, a compact test-sensor cartridge 200 is shown according to one embodiment of the present invention. The cartridge 200 in the illustrated embodiment is a generally round drum cartridge having a generally flat face 202 and a curved side portion 204. The cartridge 200 has an outer diameter ranging generally, from about 0.25 inches to about 2.0 inches and a length L generally ranging from about 0.25 inches to about 2.0 inches. More specifically, the cartridge 200 may have an outer diameter ranging from about 0.50 inches to about 1.0 inches and a length L ranging from about 0.50 inches to about 1.5 inches. The length L is generally slightly larger than the length of a test sensor 206 stored within the cartridge 200.

The cartridge 200 of the illustrated embodiment includes ten generally uniformly spaced test-sensor cavities 208, each storing a respective one of ten individual test sensors 206. The test-sensor cavities 208 may be interconnected or separate from one another. It is contemplated that the cartridge 200 may include a different number of test sensors 206 and corresponding test-sensor cavities 208. The test sensors 206 may be electrochemical, optical, colorimetric or the like. The test sensors 206 are sealed within the respective test-sensor cavities 208 to assist in preventing or inhibiting the test sensors 206 from being exposed to the atmosphere and/or moisture. The seal may be made of foil or other metallic materials or polymeric materials. FIGS. 2c,d show the cartridge 200 having foil 214 placed over the face 202 and the end 213 of the cartridge 200. The cartridge 200 may further include one or more desiccant compartments 210 for maintaining the test sensors 206 at adequate moisture levels such that accurate testing may be achieved. The cartridge 200 of the illustrated embodiment includes 10 desiccant compartments 210, each of which corresponds with a respective test-sensor cavity 208.

According to the present invention, the test-sensor cavities 208 and the corresponding test sensors 206 have a generally non-radial layout. To form the non-radial layout, the test-sensor cavities 208 are generally offset from a radial line 2-2 extending through the center of the cartridge 200 and the center of a test-sensor cavity at an angle A ranging from approximately 1.0 degree to approximately 30 degrees. More specifically, the angle A may range from approximately 10 degrees to approximately 20 degrees.

A cartridge having a non-radial layout (e.g., cartridge 200) according to the present invention provides several benefits over a cartridge adapted to store the same amount of test sensors in a radial layout (e.g., cartridge 100 of prior art). For example, the cartridge 200 provides for a greater clearance between each test-sensor cavity 208. Thus, the cartridge 200 has a greater tolerance and is therefore generally more robust and sturdy than, for example, the cartridge 100 having a radial layout. Thus, the area of the cartridge 200 between the test-sensor cavities 208 is less likely to be torn or broken, which may promote ease in manufacturing. Additionally, tears in the area between test-sensor cavities 208 may expose the test sensors 206 within the cavities 208 to potential contaminants and/or moisture in the atmosphere, which may be harmful to a user and/or lead to inaccurate test results. Furthermore, if the area between adjacent test-sensor cavities is too small, moisture may migrate from a cavity from which a sensor has been removed to the next sensor cavity via the small area between the test-sensor cavities.

According to another embodiment of the present invention, the cartridge 200 optionally includes a plurality of generally uniformly-spaced tooth-like notches 212 on an end 213 of the cartridge 200 opposite the face 202 (see FIG. 2b). The notches 212 are set off at an angle and molded onto the side portion 204 of the cartridge 200. The number of notches 212 may correspond to the number of test sensors 206 and/or test-sensor cavities 208. Thus, the cartridge 200 of the embodiment of FIG. 2b includes ten notches 212. The notches are generally adapted to be used, for example, with a mechanism for indexing and/or excising a test sensor 206 from the cartridge 200. It is contemplated that the cartridge 200 may have notches shaped differently than those shown in the illustrated embodiment. It is further contemplated that the notches may be arranged along the outer edge or circumference of the cartridge 200.

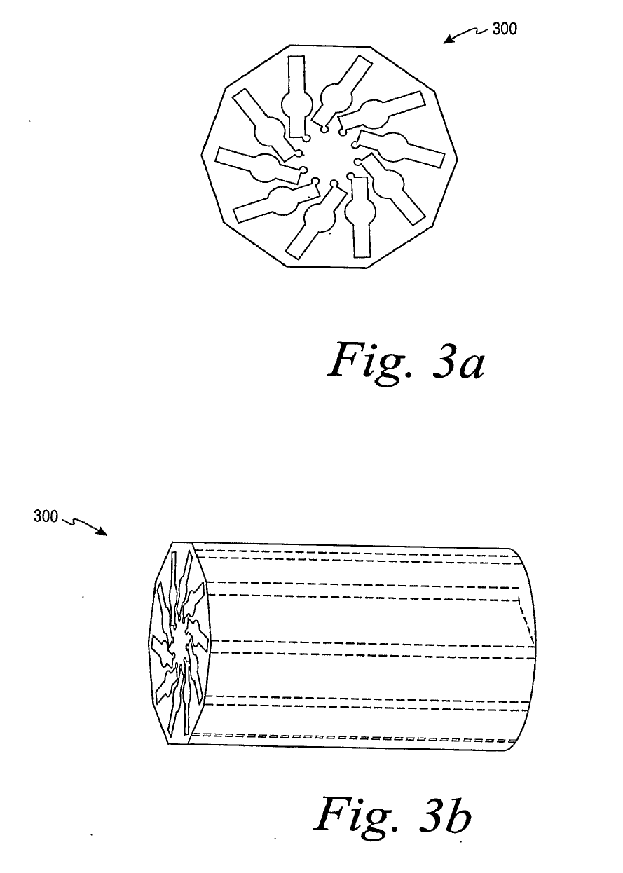

Although in the illustrated embodiments, the face 202 of the cartridge 200 is generally round, it is contemplated that the face 202 may have other shapes including, but not limited to, hexagonal, octagonal, decagonal, or other polygonal shapes. For example, FIGS. 3a,b illustrate a decagonal test-sensor cartridge 300, according to another embodiment of the present invention, having a non-radial test sensor layout.

According to one embodiment; the cartridge of the present invention (e.g., cartridge 200) includes electrochemical test sensors. The test sensors may include an enzyme. For example, if the analyte to be tested is glucose, the test sensors may contain, for example, glucose dehydrogenase or glucose oxidase.

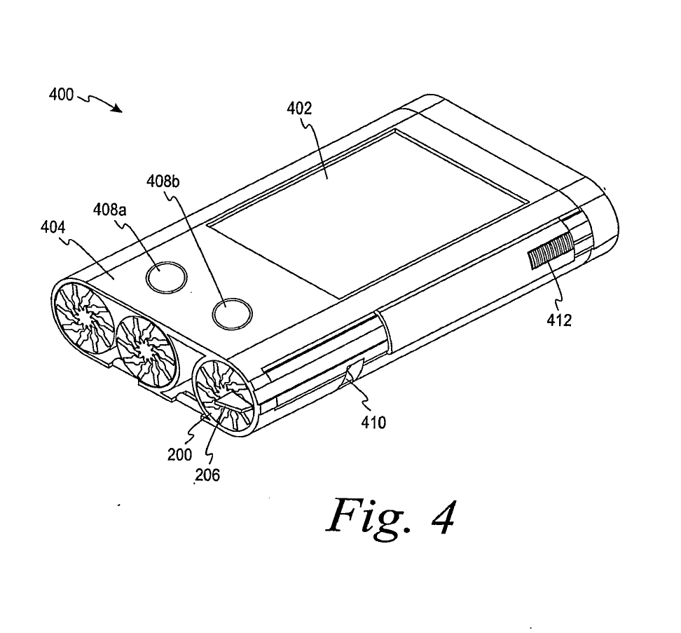

It is contemplated that one or more cartridges of the present invention may be stored within an analyte-testing instrument (e.g., a meter). Turning now to FIG. 4, an analyte-testing instrument or meter 400 is shown according to one embodiment in combination with the test-sensor cartridge 200 of FIGS. 2a,b. The meter 400 includes a display 402, a body portion 404, and a user-interface mechanism. In the embodiment of FIG. 4, the user-interface mechanism includes a plurality of buttons 408a,b. It is contemplated that a different number of buttons 408a,b may be included on the meter 400. The buttons may also have a different shape, size, and/or position on the meter 400 than the buttons 408a,b shown in FIG. 4. It is also contemplated that the user-interface mechanism may include other mechanisms suitable for communicating with the meter 400 including, but not limited to, a scroll wheel and/or a touch screen. One example of a display 402 that may be used in the meter 400 is a liquid-crystal display. The meter 400 typically shows information from a testing procedure and/or in response to signals input by the user-interface mechanism (e.g., buttons 408a,b) on the display 402 and then stores the information in memory. The result of the testing may also be announced audibly, by, for example, using a speaker, and stored in memory. The meter 400 may further include an indexing mechanism 410 for indexing the test-sensor cartridge 200 and/or an excising mechanism 412 for excising a test sensor 206 from the test-sensor cartridge 200. It is contemplated that other analyte-testing instruments and/or other test-sensor cartridges (e.g., test-sensor cartridge 300 of FIGS. 3a,b) in accordance with the present invention.

The cartridge 200 of FIGS. 2a,b is shown being stored within the meter 400. It is also contemplated that one or more additional similar cartridges may be stored as replacement cartridges within the meter 400. Storing replacement test-sensor cartridges within a meter may be desirable so that a user may carry multiple cartridges around within a single enclosure. Moreover, once the user places the replacement cartridges in the meter, the user need not concern him or herself with whether a presently in-use cartridge includes an adequate amount of unused test sensors or with remembering to bring an extra cartridge, for example, each time the user leaves the house.

Moreover, although the present invention has been described in reference to a compact cartridge, it is contemplated that the non-radial layout of the test sensors shown in FIGS. 2a,b and described above may be incorporated into any suitable test-sensor cartridge.

### Alternative Embodiment A

A test-sensor cartridge comprising a first face, a second opposing face, and a side portion connecting the first and second opposing faces, the first face forming a plurality of test-sensor cavities that is generally uniformly positioned therethrough, the plurality of the test sensor cavities being arranged in a non-radial layout, the plurality of test-sensor cavities containing a respective test sensor, the test sensor being adapted to assist in determining a concentration of an analyte.

### Alternative Embodiment B

The cartridge of Alternative Embodiment A, wherein at least the first face is generally round.

### Alternative Embodiment C

The cartridge of Alternative Embodiment B, wherein the second face is generally round.

### Alternative Embodiment D

The cartridge of Alternative Embodiment B, wherein the outer diameter of the face of the cartridge is from about 0.25 inches to about 2.0 inches.

### Alternative Embodiment E

The cartridge of Alternative Embodiment B, wherein the outer diameter of the face of the cartridge is from about 0.50 inches to about 1.5 inches.

### Alternative Embodiment F

The cartridge of Alternative Embodiment A, wherein at least the first face is generally polygonal.

### Alternative Embodiment G

The cartridge of Alternative Embodiment F, wherein the second face is generally polygonal.

### Alternative Embodiment H

The cartridge of Alternative Embodiment A, wherein the second face further comprises a plurality of generally uniformly-spaced notches, the number of notches corresponding to the number of test-sensor cavities.

### Alternative Embodiment I

The cartridge of Alternative Embodiment A wherein the test-sensor cavities are generally offset from a radial position at an angle ranging from approximately 1.0 degree to approximately 30 degrees.

### Alternative Embodiment J

The cartridge of Alternative Embodiment A wherein the test-sensor cavities are generally offset from a radial position at an angle ranging from approximately 10 degrees to approximately 20 degrees.

### Alternative Embodiment K

The cartridge of Alternative Embodiment A, wherein the test sensor is an electrochemical test sensor.

### Alternative Embodiment L

The cartridge of Alternative Embodiment K, wherein the test sensor includes an enzyme.

### Alternative Embodiment M

The cartridge of Alternative Embodiment L, wherein the analyte is glucose.

### Alternative Embodiment N

The cartridge of Alternative Embodiment M, wherein at least the enzyme is glucose dehydrogenase.

### Alternative Embodiment O

The cartridge of Alternative Embodiment M, wherein at least the enzyme is glucose oxidase.

### Alternative Embodiment P

An instrument adapted to determine an analyte concentration of a fluid sample using a test sensor, the instrument comprising:
a display adapted to display information to a user;
a user-interface mechanism adapted to allow the user to interact with the instrument;
a test-sensor cartridge comprising a first face, a second opposing face, and a side portion connecting the first and second opposing faces, the first face forming a plurality of test-sensor cavities that is generally uniformly positioned therethrough, the plurality of the test sensor cavities being arranged in a non-radial layout, the plurality of test-sensor cavities containing a respective test sensor, the test sensor being adapted to assist in determining a concentration of the analyte; and
a body portion including at least a first opening formed therein, the first opening being adapted to receive a test sensor from the test-sensor cartridge.

### Alternative Embodiment Q

The instrument of Alternative Embodiment P, wherein at least the first face is generally round.

### Alternative Embodiment R

The instrument of Alternative Embodiment Q, wherein the second face is generally round.

### Alternative Embodiment S

The instrument of Alternative Embodiment Q, wherein the outer diameter of the face of the cartridge is from about 0.25 inches to about 2.0 inches.

### Alternative Embodiment T

The instrument of Alternative Embodiment Q, wherein the outer diameter of the face of the cartridge is from about 0.50 inches to about 1.5 inches.

### Alternative Embodiment U

The instrument of Alternative Embodiment P, wherein at least the first face is generally polygonal.

### Alternative Embodiment V

The instrument of Alternative Embodiment U, wherein the second face is generally polygonal.

### Alternative Embodiment W

The instrument of Alternative Embodiment P, wherein the second face further comprises a plurality of generally uniformly-spaced notches, the number of notches corresponding to the number of test-sensor cavities.

### Alternative Embodiment X

The instrument of Alternative Embodiment P wherein the test-sensor cavities are generally offset from a radial position at an angle ranging from approximately 10 degrees to approximately 30 degrees.

### Alternative Embodiment Y

The instrument of Alternative Embodiment P wherein the test-sensor cavities are generally offset from a radial position at an angle ranging from approximately 10 degrees to approximately 20 degrees.

### Alternative Embodiment Z

The instrument of Alternative Embodiment P, wherein the test sensor is an electrochemical test sensor.

### Alternative Embodiment AA

The instrument of Alternative Embodiment Z, wherein the test sensor includes an enzyme.

### Alternative Embodiment AB

The instrument of Alternative Embodiment AA, wherein the analyte is glucose.

### Alternative Embodiment AC

The instrument of Alternative Embodiment AB, wherein at least the enzyme is glucose dehydrogenase.

### Alternative Embodiment AC

The instrument of Alternative Embodiment AB, wherein at least the enzyme is glucose oxidase.

### Alternative Embodiment AD

The instrument of Alternative Embodiment P further comprising a mechanism adapted to index the test sensor from the test-sensor cartridge.

### Alternative Embodiment AE

The instrument of Alternative Embodiment P further comprising a mechanism adapted to excise the test sensor from the test-sensor cartridge.

### Alternative Process AF

A method of excising a test sensor from a test-sensor cartridge located within an analyte-testing instrument, the method comprising the acts of:
providing a test-sensor cartridge comprising a first face, a second opposing face, and a side portion connecting the first and second opposing faces, the first face forming a plurality of test-sensor cavities that is generally uniformly positioned therethrough, the plurality of the test sensor cavities being arranged in a non-radial layout, the plurality of test-sensor cavities containing a respective test sensor, the test sensor being adapted to assist in determining a concentration of an analyte;
providing an instrument comprising a display adapted to display information to a user, a user-interface mechanism adapted to allow the user to interact with the instrument, a body portion including at least a first opening formed therein, the first opening being adapted to receive a test sensor from the test-sensor cartridge, and an excise mechanism;
moving the excise mechanism so as to excise a test sensor from the cartridge, the excised test sensor being positioned within the first opening of the instrument.

### Alternative Process AG

The method of Alternative Process AF, wherein at least the first face is generally round.

### Alternative Process AH

The method of Alternative Process AG, wherein the second face is generally round.

### Alternative Process AI

The method of Alternative Process AG, wherein the outer diameter of the face of the cartridge is from about 0.25 inches to about 2.0 inches.

### Alternative Process AJ

The method of Alternative Process AG, wherein the outer diameter of the face of the cartridge is from about 0.50 inches to about 1.5 inches.

### Alternative Process AK

The method of Alternative Process AF, wherein at least the first face is generally polygonal.

### Alternative Process AL

The method of Alternative Process AK, wherein the second face is generally polygonal.

### Alternative Process AM

The method of Alternative process AF, wherein the second face further comprises a plurality of generally uniformly-spaced notches, the number of notches corresponding to the number of test-sensor cavities.

### Alternative Process AN

The method of Alternative Process AF wherein the test-sensor cavities are generally offset from a radial position at an angle ranging from approximately 1.0 degrees to approximately 30 degrees.

### Alternative Process AO

The method of Alternative Process AF wherein the test-sensor cavities are generally offset from a radial position at an angle ranging from approximately 10 degrees to approximately 20 degrees.

### Alternative Process AP

The method of Alternative Process AF, wherein the test sensor is an electrochemical test sensor.

### Alternative Process AQ

The method of Alternative Process AP, wherein the test sensor includes an enzyme.

### Alternative Process AR

The method of Alternative Process AQ, wherein the analyte is glucose.

### Alternative Process AS

The method of Alternative Process AR, wherein at least the enzyme is glucose dehydrogenase.

### Alternative Process AT

The instrument of Alternative Process AR, wherein at least the enzyme is glucose dehydrogenase.

### Alternative Process AU

The method of Alternative Process AF further comprising a mechanism adapted to index the test sensor from the test-sensor cartridge.

While the invention is susceptible to various modifications and alternative forms, specific embodiments and methods thereof have been shown by way of example in the drawings and are described in detail herein. It should be understood, however, that it is not intended to limit the invention to the particular forms or methods disclosed, but, to the contrary, the intention is to cover all modifications, equivalents and alternatives falling within the spirit and scope of the invention as defined by the appended claims.

1. A test-sensor cartridge comprising a first face, a second opposing face, and a side portion connecting the first and second opposing faces, the first face forming a plurality of test-sensor cavities that is generally uniformly positioned therethrough, the plurality of the test sensor cavities being arranged in a non-radial layout, the plurality of test-sensor cavities containing a respective test sensor, the test sensor being adapted to assist in determining a concentration of an analyte.

2. The cartridge of claim 1, wherein at least the first face is generally round.

3. The cartridge of claim 2, wherein the second face is generally round.

4. The cartridge of claim 2, wherein the outer diameter of the face of the cartridge is from about 0.25 inches to about 2.0 inches.

5. (canceled)

6. The cartridge of claim 1, wherein at least the first face is generally polygonal.

7. (canceled)

8. The cartridge of claim 1, wherein the second face further comprises a plurality of generally uniformly-spaced notches, the number of notches corresponding to the number of test-sensor cavities.

9. The cartridge of claim 1, wherein the test-sensor cavities are generally offset from a radial position at an angle ranging from approximately 1.0 degree to approximately 30 degrees.

10-11. (canceled)

12. The cartridge of claim 1, wherein the test sensor includes an enzyme.

13. The cartridge of claim 12, wherein the analyte is glucose.

14. The cartridge of claim 13, wherein the enzyme is glucose dehydrogenase.

15. (canceled)

16. An instrument adapted to determine an analyte concentration of a fluid sample using a test sensor, the instrument comprising:
a display adapted to display information to a user;
a user-interface mechanism adapted to allow the user to interact with the instrument;
a test-sensor cartridge comprising a first face, a second opposing face, and a side portion connecting the first and second opposing faces, the first face forming a plurality of test-sensor cavities that is generally uniformly positioned therethrough, the plurality of the test sensor cavities being arranged in a non-radial layout, the plurality of test-sensor cavities containing a respective test sensor, the test sensor being adapted to assist in determining a concentration of the analyte; and
a body portion including at least a first opening formed therein, the first opening being adapted to receive a test sensor from the test-sensor cartridge.

17. The instrument of claim 16, wherein the first face and the second opposing face are generally round.

18-22. (canceled)

23. The instrument of claim 16, wherein the second face further comprises a plurality of generally uniformly-spaced notches, the number of notches corresponding to the number of test-sensor cavities.

24. The instrument of claim 16, wherein the test-sensor cavities are generally offset from a radial position at an angle ranging from approximately 1.0 degrees to approximately 30 degrees.

25-30. (canceled)

31. The instrument of claim 16, further comprising a mechanism adapted to index the test sensor from the test-sensor cartridge.

32. The instrument of claim 16, further comprising a mechanism adapted to excise the test sensor from the test-sensor cartridge.

33. A method of excising a test sensor from a test-sensor cartridge located within an analyte-testing instrument, the method comprising the acts of:
providing a test-sensor cartridge comprising a first face, a second opposing face, and a side portion connecting the first and second opposing faces, the first face forming a plurality of test-sensor cavities that is generally uniformly positioned therethrough, the plurality of the test sensor cavities being arranged in a non-radial layout, the plurality of test-sensor cavities containing a respective test sensor, the test sensor being adapted to assist in determining a concentration of an analyte;
providing an instrument comprising a display adapted to display information to a user, a user-interface mechanism adapted to allow the user to interact with the instrument, a body portion including at least a first opening formed therein, the first opening being adapted to receive a test sensor from the test-sensor cartridge, and an excise mechanism;
moving the excise mechanism so as to excise a test sensor from the cartridge, the excised test sensor being positioned within the first opening of the instrument.

34-39. (canceled)

40. The method of claim 33, wherein the second face further comprises a plurality of generally uniformly-spaced notches, the number of notches corresponding to the number of test-sensor cavities.

41. The method of claim 33, wherein the test-sensor cavities are generally offset from a radial position at an angle ranging from approximately 1.0 degrees to approximately 30 degrees.

42-47. (canceled)

48. The method of claim 33, further comprising a mechanism adapted to index the test sensor from the test-sensor cartridge.
